(19) United States
(12) Patent Application Publication
(10) Pub. No.: US 2009/0054853 A1
(43) Pub. Date: Feb. 26, 2009

(54) SURGICAL TOOL SYSTEM THAT FORMS A STERILE GAS BARRIER AT THE SITE AT WHICH THE TOOL OF THE SYSTEM IS USED AND THAT ACTIVATES ENERGY-ACTIVATED AGENTS DISCHARGED AT THE SITE

(76) Inventors: Richard F. Huyser, Kalamazoo, MI (US); Douglas Tyler, SR., Paw Paw, MI (US); David S. Goldenberg, Mattawan, MI (US)

Correspondence Address: INTEL. PROP./ RND;STRYKER CORPORATION, 4100 EAST MILHAM AVE., KALMAZOO, MI, 49001-6197, US

(21) Appl. No.: 12/195,868

(22) Filed: Aug. 21, 2008

Related U.S. Application Data
(60) Provisional application No. 60/957,214, filed on Aug. 22, 2007.

(51) Int. Cl. A61M 25/00; A61B 17/00

(52) U.S. Cl. 604/264; 606/205

(57) ABSTRACT

A surgical tool system including a tool such as a retractor, forceps or a power activated tool. The system also includes a source of gas and a means for inducing the flow of a agent that has a therapeutic effect into the gas stream. The tool has a bore into which the gas stream is introduced. The tool also has a discharged port from which the gas stream is discharged toward the site to which the tool is applied. The gas plume that is discharged from the tool forms a barrier that prevents airborne contaminates from reaching the site to which the tool is applied. The tool also has a device for emitting energy separate from any energy the tool emits to perform its function. This energy emitter emits energy that activates the therapeutic agent discharge from with the gas.

### RELATIONSHIP TO EARLIER FILED APPLICATION

This application claims priority from U.S. Provisional Patent App. No. 60/957,214 filed 22 Aug. 2007, the contents of which are incorporated herein by reference.

### FIELD OF THE INVENTION

This invention relates generally to surgical tools and, more particularly, to surgical tools capable of forming a sterile gas barrier at the sites at which they are used. This invention also relates to surgical tools capable of delivering and/or activating photosensitive agents present or delivered at the sites at which the tools are used.

### BACKGROUND OF THE INVENTION

To perform many medical and surgical procedures, it is necessary to expose the internal body tissue on which the procedure is to be performed to the ambient environment. An inevitable result of taking this action is that it exposes the tissue to contaminates in the ambient atmosphere. Sometimes, this contaminate-on-tissue contact results in patient infection.

A number of different means are presently employed to minimize the extent to which the internal body tissue and organs are exposed to these airborne contaminates. Often, integral with the room or suite in which the procedure is to be performed is a ventilation system. This ventilation system is provided with filters that trap the contaminates prior to the air being flowed into or recirculated back into the room/suite. Also, ideally, the air is cooled prior to being flowed into the room/suite. These ventilation systems do reduce the extent to which infection-causing contaminates are contained within the air present in an operating room/procedure suite. However, practically, these systems cannot eliminate all contaminates. Also, this type of system projects an air stream onto both the medical professionals and the adjacent tools and instruments. These bodies and objects disrupt the air flow. This disruption can cause the air flow to become turbulent. As discussed below, such turbulence can reduce the effectiveness of the desired barrier.

Furthermore, this system typically includes some type of filter. If the filter is not properly maintained, the air quality could be subjected to degradation. Also, poor maintenance of the filter could result in the introduction of a turbulent air flow into the operating room/suite. This type of flow could result in an air flow that actually induces the flow of contaminates.

Systems have been provided that form gas barriers around surgical sites. One version of this type of system includes a blower unit, essentially a fan, that is placed in close proximity to the portion of the body in which the opening into the patient is formed. The blower unit outputs a laminar flow of sterile air over the surface of the body, over the surgical site. Contaminates that precipitate out of the ambient air above the surgical site, are entrained in this air flow. This air flow thus functions as a barrier that prevents these contaminates from reaching exposed tissue. A disadvantage of this type of system is that it requires the placement of a very large device, the blower unit, in close proximity to the patient. Moreover, given the volume of air this type of unit discharges over the patient and the distance of the unit from the patient, there may be situations wherein this unit actual entrains contaminates from the ambient environment and draws them over the surgical site.

Other systems that form a sterile gas barrier around a surgical site include components designed for closer placement around or even at the site. One such version of this type of system includes a planer, flexible mat-like device. The device includes a center opening and is positioned so that the opening is disposed over the surgical site. Air flows across the device, across the opening, to form a sterile gas barrier above the surgical site. Still another version of this type of device includes a wand placed in the opening in the body through which access to the surgical site is obtained. The wand is formed so that, at the distal end, there are one or more openings. Gas discharged from the distal end of the wand forms a high pressure plume in and immediately above the surgical site. (“High pressure” here is understood to be greater than the pressure of the ambient air in the operating room.) This plume of air prevents contaminates in the surrounding ambient air from precipitating into the surgical site. In some implementations of this system, CO₂ or CO₂-enriched air is the gas discharged from the device. CO₂ is denser than ambient air. Therefore, the resultant plume forms an enhanced barrier around the site. Also, for cardiac procedures, the introduction of CO₂ reduces the likelihood of an air embolism.

One common feature of the above systems is that they require the placement of a new component, the gas mat or the wand, at or near the surgical site. This component adds to the clutter of medical devices around the surgical site. Adding to this clutter is of course one thing medical professionals would prefer to avoid since it adds to the complexities of the medical/surgical procedure.

### SUMMARY OF THE INVENTION

This invention relates to a new and useful surgical tool system. Surgical tools of this system are designed to perform two tasks. First, a tool of this invention has components that enable it to work the tissue at the surgical site to which they are applied. The surgical tools of the system of this invention are also designed to form a sterile gas barrier adjacent the tissue to which they are applied.

Specifically, a tool of this invention has a physical shape that enables the tool to perform a specific surgical procedure. For example, a retractor of the surgical tool system of this invention is able to hold tissue away from a surgical site. Forceps of this invention are able to grasp tissue. A tool of this invention also is formed with a conduit and one or more openings. Sterile gas is flowed to the tool. In some versions of the invention, this gas is from a filter, also part of the invention. Alternatively, the gas is from a self-contained source, again, the source being part of the invention. The gas is vented through the openings integral with the tool. The vented gas functions as either a gas stream over or a gas plume rising within and away from the surgical site. In either situation, this gas flow functions as a barrier that prevents contaminates in the surrounding ambient atmosphere from precipitating onto the exposed tissue at the surgical site.

The system of this invention is also capable of dispersing medical agents including photo-activated agents. Tools of this invention are also provided with light emitting elements. These light emitting elements, when actuated, emit light that activates the photo-activated agents.

### BRIEF DESCRIPTION OF THE DRAWINGS

The invention is pointed out with particularity in the claims. The above and further features and advantages of this invention are better understood by reference to the following Detailed Description taken in conjunction with the accompanying drawings in which:

FIG. 1 is a block diagram of the components of the surgical tool system of this invention;

FIG. 2 perspective view looking upwardly of one surgical tool, a retractor, integral with the tool system of this invention;

FIG. 3 is a cross sectional view of the retractor of FIG. 2;

FIG. 4 illustrates how the retractor of this invention is used to simultaneously expose a surgical site, provide a sterile gas barrier at the site and provide a light used to activate an photosensitive pharmaceutical agent present at the site;

FIG. 5 is a perspective view of an alternative surgical tool, an alternative retractor, of this invention;

FIG. 6 illustrates how the retractor of FIG. 5 is used to both hold an incision open and create a laminar gas barrier above the opening formed by the incision;

FIG. 7 is a block diagram of an alternative system of this invention;

FIG. 8 is a side view of the tool, the suction wand, integral with the system of FIG. 7;

FIG. 9 is plan view, looking downwardly on the suction wand;

FIG. 10 is a part block and part perspective diagram of another alternative system of this invention;

FIG. 11 is a cross sectional view illustrating how the tool of FIG. 10 may be positioned at a surgical site, specifically within a medullary canal;

FIG. 12 illustrates how the tool of FIG. 10 is used over a surgical site;

FIG. 13 illustrates another alternative tool of this invention;

FIG. 14 is a cross sectional view of the distal end of another tool of this invention.

### DETAILED DESCRIPTION

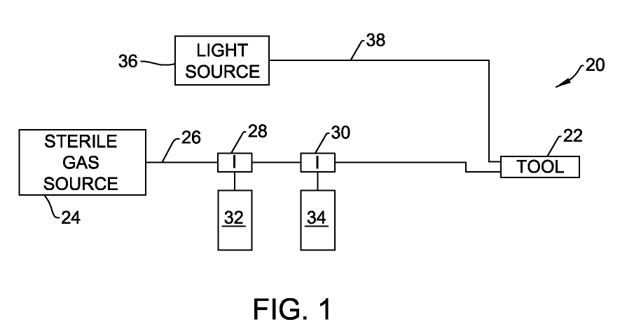

The basic components of a surgical tool system 20 of this invention are seen in FIG. 1. System 20 includes a tool 22 designed to perform a specific function during a medical or surgical procedure. Examples of such tools include, but are not limited to: retractors; forceps; and clamps. Other tools of this invention may include power tools such as motorized handpieces; laser cutting or coagulation tools and tools; RF cutting, coagulation; irrigation; or ablation tools; and tools that emit ultrasonic energy to a target site. Often the above types of tools include some type of “accessory” that applies the output energy (mechanical, photonic, electromagnetic or ultrasonic) to the tissue. For the purpose of this invention, this accessory is considered part of the tool.

System 20 also includes a sterile gas source 24. In some versions of the invention, the sterile gas source includes one or more canisters that contain air, a single gas, or a blend of gases that have been filtered to remove contaminates. In some embodiments of this version of the invention, the canister is filled with CO₂. Thus, depending on the construction, gas source 24 may emit pure CO₂, a gas blend that includes percent CO₂ beyond that normally present in the atmosphere. In some versions of the invention, gas source 24 includes a device that draws in ambient air and filters the air to produce an essentially contaminate free air stream. This filtration unit may not be located in the operating room but in a central location in the medical facility in which the system 20 is employed. In some versions of the system, gas source 24 comprises a hybrid of the two systems. A filtration unit provides a stream of contaminate free air; a canister provides sterile CO₂ so the result gas stream is a CO₂-enriched stream of sterile air. Thus, it should be understood that the exact structure of the sterile gas source 24 may vary from what has been described.

The gas output by source 24 is discharged to tool 22 through a gas line 26. First and second inductors 28 and 30, are connected to gas line 26 so as to be located between the gas source 24 and tool 22. First inductor 28 is connected to a container 32 that holds sterile water. Second inductor 30 is connected to a container 34 that holds a pharmaceutical agent.

System 20 also includes a light source 36. Light source 36 emits light of a specific wavelength. More particularly the light is emitted at a wavelength suitable for activating the pharmaceutical agent. The actual light emitting assembly internal to the source may be a tuned laser or a set of diodes that emit light at the appropriate wavelength. Alternatively, the light source may contain a light emitting device that emits light at a plurality of wavelengths and filters that only pass through light at the selected wavelength. Thus, there may be appreciable variations in the structure of the light source integral with this invention. The light emitted from source 36 discharged to tool 24 by a fiber-optic cable 38.

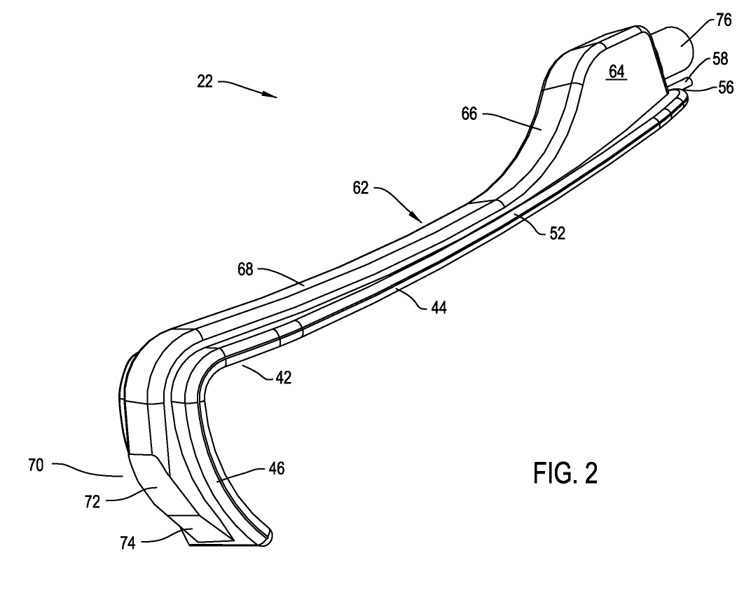

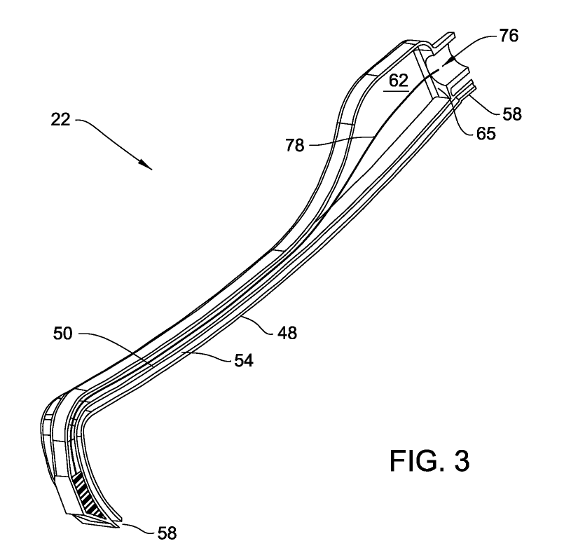

FIGS. 2 and 3 illustrate the structure of one particular tool 22 of system 20 of this invention. Tool 22 is a retractor. The retractor 22 has a main body 42 that is the shape of a conventional retractor. That is, the main body has a elongated leg 44. At the distal end of the leg 44, the body 42 curves downwardly to define a foot 46. (“Distal” is understood to mean away from the medical practitioner and towards the body site at which the procedure is performed. “Proximal” means towards the medical practitioner and away from the body site.)

Retractor 22 of this invention is further formed so that the main body 42 is hollow. Thus, the main body has spaced apart lower and upper panels 48 and 50, respectively. Outwardly curved side panels 52 (one shown) extend between the lower and upper panels 48 and 50, respectively. Panels 48, 50 and 52, collectively define a conduit 54 through the retractor main body 42. At the proximal end, an end panel 56 that extends between the upper and lower panels 48 and 50, respectively, and between the side panels 52. The end panel 56 effectively closes the distal end of conduit 54. A tubular inlet spike 58 extends proximally rearward from the end panel 56. Inlet spike 58 is dimensioned to receive the distal end of gas line 26.

Retractor 22 is further formed to have hollow rib 62 that is disposed over the top of the main body 44. Generally, along the length of the retractor 22, rib 62 has a width less than that of retractor main body 44. Adjacent the proximal end of the retractor 22, rib 62 has a tail 64 with a relatively large height, measured from the top of the main body 44. At the most proximal end of rib 62, end wall 65 extends downwardly from top of the tail 64 to the retractor upper pane 50. Extending distally, the height of the rib 62 decreases to provide the rib with a curved transition section 66 immediately distal from the tail 64. Rib transition section 66 leads to a main section 68 with a height approximately 10 to 25% of the tail 64. Rib main section 68 extends over the 40 to 70% of the retractor main body leg 44 and over the curve that forms that forms the transition from the leg to the foot 46 and over the foot. The rib main section 68 terminates a short distal proximally rearward of the distal end terminus of the retractor main body foot 46.

Seated in and flush with the distal end of rib main section 68 is a lens 70 with one or more facets. The lens 70 is disposed over the outer surface of the retractor main body foot 46 to form the most distal portion of the rib 62. In the illustrated version of the invention, lens 70 has two exposed faces, facets 72 and 74. Facet 72 is generally parallel and located forward of the section of the retractor upper panel 50 that forms the retractor foot 46. Facet 74 angles downwardly from facet 72 to abut the immediately underlying retractor upper panel 50.

Rib 62 is hollow to allow the transmission of light from light source 36 to lens 70. In the illustrated version of the invention, a connector 76 extends proximally rearward from the rib end wall 65. Connector 76 is dimensioned to receive fiber optical cable 38. In some versions of the invention, the inner surfaces of the panels forming the retractor rib 62 are formed with a reflective material to facilitate the downline transmission of light to lens 70. In alternative versions of the invention, a fiber optic cable, represented as line 78, extends from connector 76 to lens 70. In some other versions of the invention, instead of rib 62 being hollow, it is formed from a solid optically transmissive material such as a transparent plastic. The outer surface of this type of rib may be coated with a reflective material to facilitate the downline transmission of light.

To ready retractor 22 of this invention for use, gas line 26 and fiber optic cable 38 are connected to, respectively, inlet spike 58 and cable connector 76.

Retractor 22 then is used to perform the function for which it is designed. Specifically, once an incision is formed to gain access to a surgical site, the retractor foot 46 is inserted in the opening defined by the incision. The opening can be widened by urging the retractor leg 44 away from the incision. Once the portal to the surgical site is defined, the retractor is held in position so that that retractor holds the portal open.

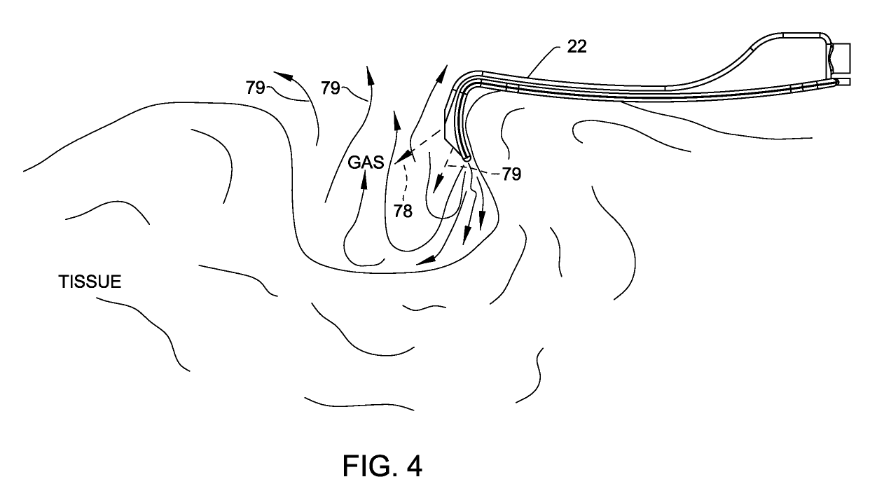

Once the retractor is so positioned, seen in FIG. 4, sterile gas is flowed from source 24. aerosolized water is introduced into the gas stream by inductor 28. An aerosolized pharmaceutical agent is introduced into the gas stream by inductor 30. The fluid stream, the sterile gas with additives, is introduced through gas line 26 into the retractor main body 42. This fluid stream flows through the retractor and is discharged out of port 58 at the free end of the foot 46. The gaseous component of the fluid stream discharged from the retractor initially fills the void space in the patient created by the incision. Then, the gas moves upwardly out of the incision. This flow is represented in FIG. 4 by arrows 79. This head of dispersing gas thus creates a barrier immediately above the incision. More specifically, the gas stream traps small sized particulate matter that could potentially contaminate the surgical site. This material, instead of falling on the tissue exposed by the incision, is entrained in the gas flow that moves away from the surgical site.

The fluid stream discharged from the retractor 22 also includes the aerosol sized water droplets of water and the pharmaceutical agent. At least a fraction of both of these compounds precipitates out of each parcel of gas before the parcel moves away from the incision. The water droplets moisturize the exposed tissue. This slows the drying out of the tissue that can otherwise occur when exposed to the relatively cold and low humidity environment of the operating room. The pharmaceutical agent performs the intended therapeutic affect on the tissue that it contacts. In some configurations of the invention, the pharmaceutical agent is an antimicrobial agent selected to neutralize any infection causing species on contaminates that might come to rest on the exposed tissue.

In some versions of the invention, the pharmaceutical agent is light activated. In these versions of the invention, light source 36 is activated. More particularly, the light source is activated to emit light at a wavelength suitable for causing the activation of the pharmaceutical agent. This light is emitted towards the tissue as represented by dashed arrows 78 emanating from the facets of lens 70

Thus, tools and instruments of surgical tool system 20 of this invention do more than perform the basic tasks for which they are designed. A surgical tool of this invention also discharges a fluid stream at the surgical site at which the tool is used. By volume, the largest component of this fluid stream is a gas. This gas, as a consequence of its filling the void space above the surgical site and diffusion above the site, forms a barrier that inhibits the extent to which contaminates in the ambient atmosphere are able to precipitate out of and land on the exposed tissue. Water and any pharmaceutical agents the fluid stream discharged from the tool do precipitate out of this fluid flow and do land on the tissue. The water inhibits the drying of this not-normally-exposed to atmosphere tissue. The pharmaceutical agent performs its intended function.

In versions of the invention wherein the pharmaceutical agent is light activated, the tool of this invention also serves as the device that emits the light required to activate the agent.

Thus, system 20 of this invention prevents exposed tissue from being exposed to airborne contaminates, keeps the tissue moist to counter act the tissue's exposure to air, and coats the tissue with pharmaceutical agent all with the aid of tool the use of which is already required. Should the pharmaceutical agent be photoactived, the tool of this invention is also capable of emitting the light needed to cause the agent to have therapeutic effect. By performing all these functions with a single tool, the clutter of bringing plural tools to the surgical site is eliminated.

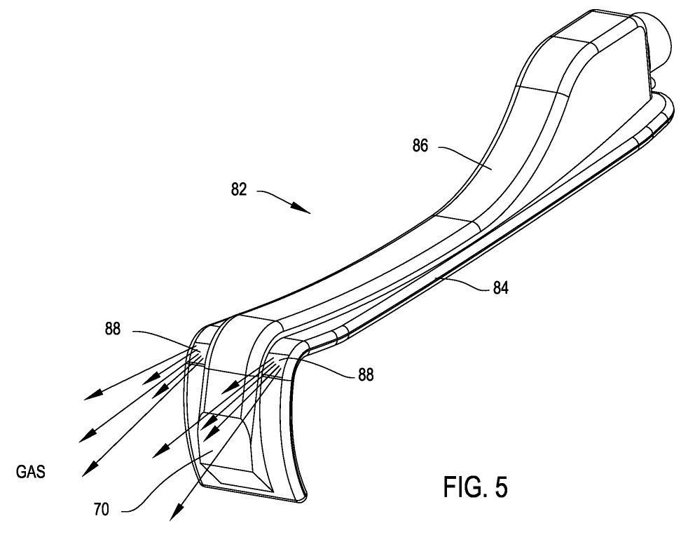

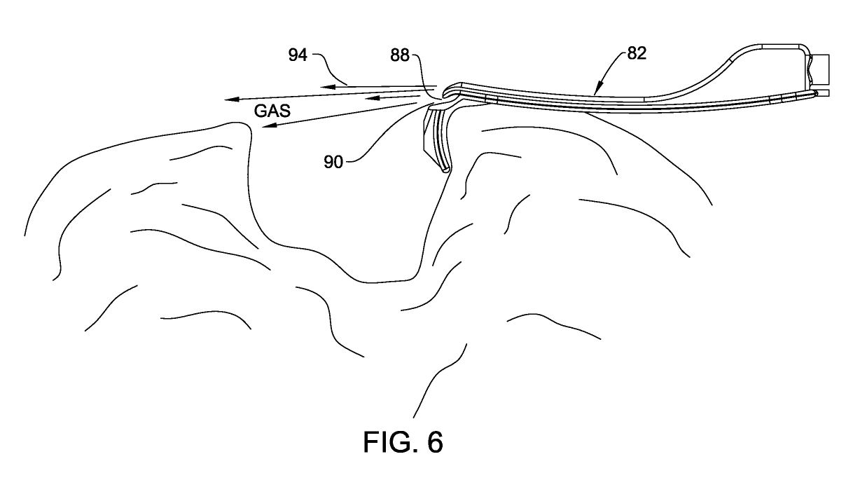

Variations in the system of this invention are possible. For example, FIGS. 5 and 6 illustrates an alternative tool 82 of this invention. Tool 82, like tool 22, is a retractor. Tool 82 has a main body 84 that is similar to main body 44 of tool 22. Tool 82 also has a rib 86 like rib 62. Tool 82 is formed so that main body 84 has two ports 88 in the upper panel of the body that are axially aligned with the conduit internal to the leg of the body. Internal to the retractor main body 84 is a baffle plate 90, seen in FIG. 6. The baffle plate 90 is located immediately below the bottom of ports 88. The baffle plate 90 thus directs the fluid stream introduced into the retractor main body 84 out through ports 88.

Thus, when retractor 82 of this invention is used the fluid stream is discharged in a linear path across the opening defined by the incision. Arrows 94 of FIG. 6 illustrate this flow. Contaminates that precipitate out of the ambient atmosphere towards the exposed tissue become entrained in this gas stream and flow away from the open surgical site. Aersolized water or pharmaceutical agents entrained in the fluid stream discharged from the retractor 82, because they are substantially heavier than the contaminates, do precipitate out of the fluid stream and fall onto the exposed tissue.

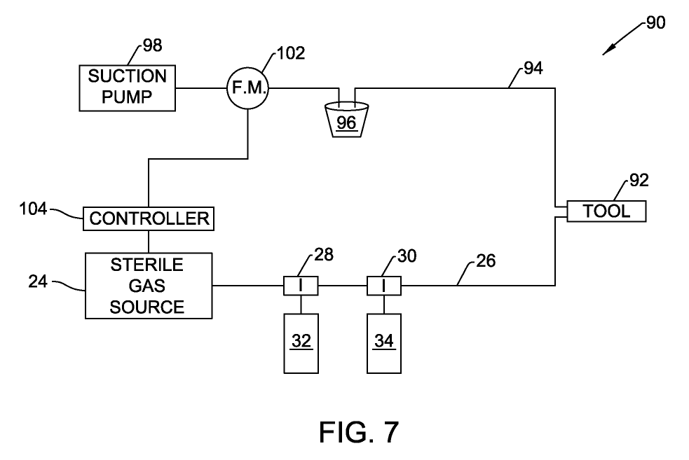

An alternative surgical tool system 90 is now initially described with initial reference to FIG. 7. The tool integral with system 90 is a suction wand 92. System 90 includes the previously described gas source 24 and gas line 26. A suction line 94 extends proximally from the suction wand 92 to a suction pump 98. Typically, a collection unit 96 is attached to the suction line 94 between the wand 92 and the pump 98. The exact structure of the collection unit 96 and 98 is not relevant to the nature of this invention.

Shown integral with the suction line 94 between the collection unit 96 and the pump 98 is a flow monitor 102. Flow monitor 102 is configured to assert a signal to the gas source 24 when the monitor detects a suction is being drawn through the wand 92. More particularly, this signal is asserted to a controller 104.

Controller 104 is integral with gas source 24 that regulates the discharge of gas from the source. Controller 104 may be a set of discrete components, a PLA or a microcontroller.

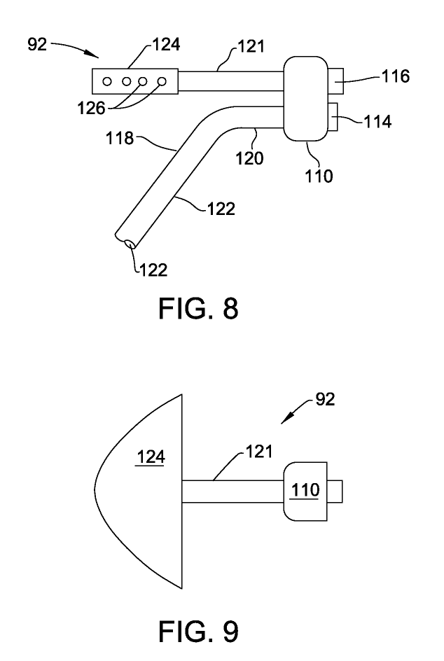

FIGS. 8 and 9 illustrate the structure of wand 92. At the proximal end, wand 92 is shaped to have head 110. Head 110 is formed with a suction fitting 114 and a gas fitting 116. Suction fitting 114 is dimensioned to receive suction line 94. Gas fitting 116 is formed to receive gas line 26. Relative to gravity and normal use of wand 92, gas fitting 116 is located above the suction fitting 114.

Extending forward from the suction fitting 114, wand 92 has a rigid suction tube 118. Suction tube 118 has a proximal section 120 that is generally axially aligned with the suction fitting. Forward from the proximal section 120 suction tube 118 is shaped to have a distal section 122 that is angled downwardly relative to the proximal section. Suction tube distal section 120 has at least one distal end opening 122. Opening 122 is the opening through which fluids are drawn from the surgical site.

Extending forward from and axially aligned with gas fitting 116 is discharge tube 121. The discharge tube 121 extends a relatively small distance forward of the gas fitting 116. In the illustrated version of the invention, the discharge tube extends forward a distance approximately equal to that of the suction tube proximal section 120. A fan-shaped spray nozzle 124 is attached to the distal end of the discharge tube. Nozzle 124 includes a number of different arcuately spaced apart openings 126. Nozzles 124, including openings 126, are constructed so that the fluid stream discharged from the nozzle is discharged in a fan pattern over the suction tube distal section 120.

Gas source controller 104 of system 90 of this invention is configured to at least partially regulate the output of gas as a function of the suction flow through the system. In some configurations of system 90, the gas source is normally off. In response to receipt of the signal that there is suction flow through tube 118 and line 94, controller 104 actuates the source 24 so there sterile gas is discharged. In some configurations of the system 90, the gas source 24 is set to always be on. In these configurations, when controller 104 receives the signal that there is suction flow, the controller boosts the rate of gas delivery.

To use wand 92, gas line 26 is connected to gas fitting 116 and suction line 94 is connected to the suction fitting 114. The wand 92 is located so that the distal end of the suction tube distal section 122 is positioned at the surgical site at the location at which the suction is to be drawn. As mentioned above, in some configurations, system 90 is set so that the fluid stream is discharged from the nozzle 124 regardless of the on/off state of the suction.

When a suction is drawn, this flow is detected by monitor 102. Monitor 102, in turn, asserts the flow detected signal to controller 104. Depending on the configuration of the system 104, in response to this signal, controller 104 either actuates the gas source or boosts the output of the fluid stream.

Thus, system 90 of this version of the invention, outputs a laminar flow of sterile gas above the location at which the suction tube draws material into the suction line 94. This air flow entrains contaminates in the ambient atmosphere that otherwise, due to the suction applied to the surgical site, would be drawn down to the exposed tissue. In one configuration of this system, this sterile gas barrier is always present, regardless of the suction on/off state. It is then boosted when the suction would otherwise cause the barrier to be disrupted. Alternatively, system 90 is configured so that the barrier is only formed when the suction is drawn.

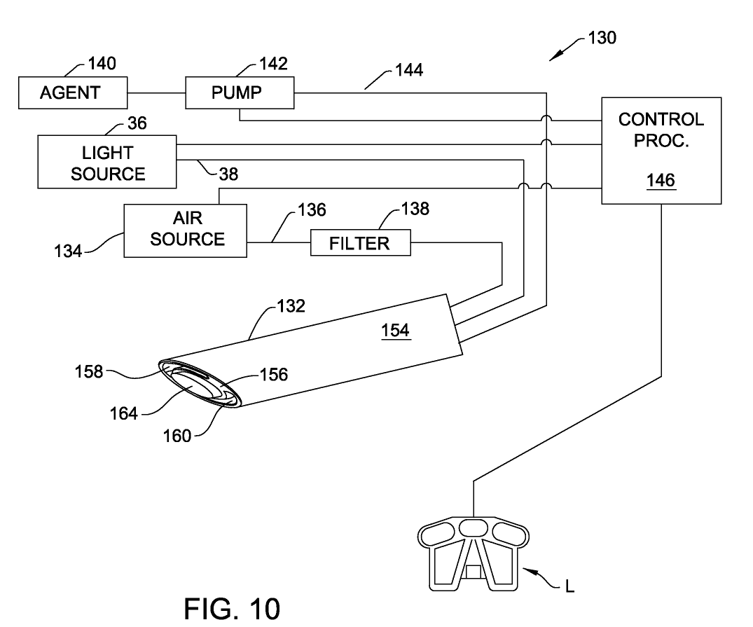

FIG. 10 illustrates another alternative system 130 of this invention with an alternative wand 132 for forming a sterile air barrier. System 130 includes a air source 134 for supplying air to wand 132 through an air line 136. Internal to air line 136 is a filter 138. Filter 138 contains material capable of removing contaminates that carry viral and bacterial sized matter out of the air stream discharged from source 134. In some embodiments of this version of the invention, wand 132 and air line 136, including filter 138, are formed as a common, single, use-once assembly. An advantage of this arrangement is that each time a new wand 132 is installed as part of the system 110, a clean airline 136 and new filter 138 is likewise installed.

System 130 also includes light source 36 and fiber optic cable 38.

In FIG. 10, element 140 is a container that stores a pharmacological agent. A pump 142 draws the agent from the container and forces it through a downline tube 144. Tube 144 is connected to the proximal end of wand 132.

Light source 36, air source 134 and pump 142 are activated based on signals received from a common control processor 146. The practitioner indicates which of these three sub-assemblies are to be activated by the entry of commands to the control processor 146. A footswitch assembly 148 may be used to input commands to processor 146. The individual pedals of the footswitch assembly are programmed to function as the on/off switch for a particular one or combination of the assemblies. Depending on factors such as practitioner preference, each footswitch pedal can be programmed so that only when depressed the associated subsystem is actuated. Alternatively, the footswitch can be programmed to press once for on and second time to shut off.

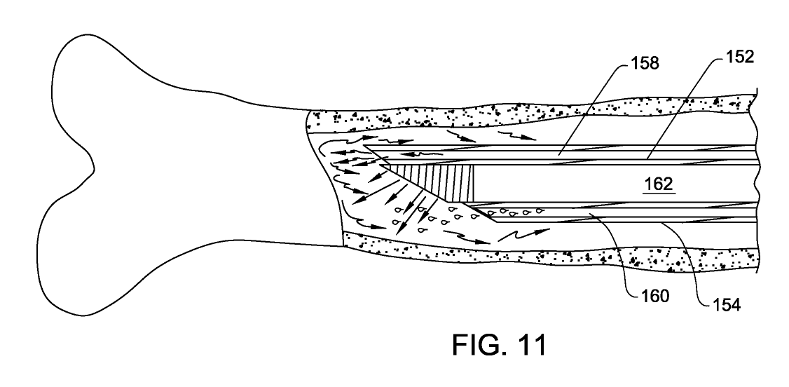

From FIGS. 10 and 11 it can be seen that wand 132 is formed to have inner and outer tubes 152 and 154, respectively. Two webs 156, one shown in FIG. 10, that are symmetrically arranged around the longitudinal axis of the wand extend between the tubes so as to suspend inner tube 152 in outer tube 154. Collectively tubes 152 and 154 and the webs 156 define two parallel channels 158 and 160 internal to the wand. Channel 158 functions as the conduit through which the filtered air from source 134 flows through the wand 132. Channel 160 functions as the conduit through which the pharmaceutical agent is discharged.

Disposed in inner tube 152 is a fiber optic core 162. The distal end of cable 38 is connected to wand 132 so that the light emitted by the cable is transmitted distally through core 162. In the illustrated version of the invention the distal end of wand has a face that is at an obtuse angle to the longitudinal axis of the wand. Thus channel 158 has an opening to the environment that is spaced forward of channel 160. Core 162 has a distal end face 164 that extends diagonally between the openings integral with channels 158 and 160.

As seen by FIG. 11, wand 132 of this invention can be inserted in narrow sites within the body, here within the medullary canal. By selective activation of the subsystems of system 130, the wand can be used to: discharge sterile air to prevent the entry of contaminates in the enclosed spaced where inserted; discharge a pharmaceutical agent; and/or emit the light needed to activate the agent.

While not illustrated, it should be understood that in some embodiments of the invention, wand 132 may be formed to have bristles that project outwardly from the outer surface of the outer tube 154. Often, but not always, these bristles are located around the distal end of the wand. In these embodiments of the invention, wand 132 thus functions as a brush for cleaning material away from the surgical site. One such brush is a femoral canal brush. Here, the bristles are used to remove bone chips and fat away from the inner surface of the bone that defines the canal. Thus, in this embodiment of the invention, wand 130, in addition to serving as a brush that removes unwanted material, also serves as the device that delivers sterile gas adjacent the tissue to prevent contaminants from contacting the tissue.

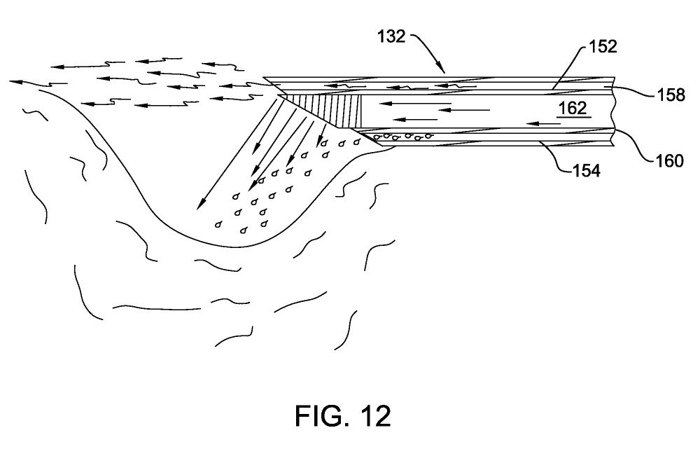

Alternatively, as seen in FIG. 12, by placing wand 132 at the edge of an incision, the air discharged from channel 158 may form a laminar air sterile air barrier over the surgical site. Owing to the diagonal profile of the face of the wand 132, both the pharmacological agent and light are emitted directly into the opening into the surgical site.

While not shown, in this version of the invention, a foot may project downwardly from the outer tube 154. In these embodiments of the invention, wand 132 functions as a retractor for maintaining access to the surgical site.

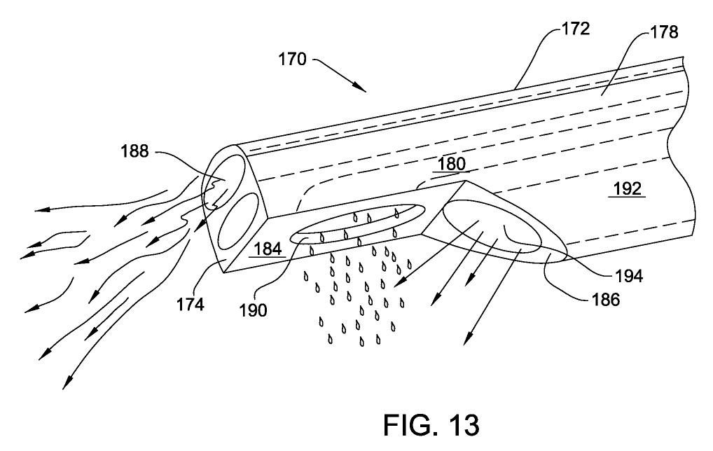

The distal end of another wand 170 is now described by reference to FIG. 13. Wand 170 is formed to have an elongated body 172. A face 174 perpendicular to the longitudinal axis of body 172 forms the distal end of the wand 170. Below face 174, wand is formed to have a pair of proximally extending undercut surfaces. The most distal surface, surface 184, extends perpendicularly proximally rearward from face 174. Undercut surface 186, the more proximal of the two surfaces, extends diagonally downward from surface 184.

Internal to wand body 172 are two longitudinally extending channels 178 and 180 both shown in phantom. Channel 178, relative to gravity, the upper of the two channels extends to an opening 188 formed in distal end face 178. Channel 178 is the channel through which the sterile gas is flowed through the wand 170. Channel 180, the lower of the two channels terminates at an opening 190 formed in undercut surface 184. Channel 180 is the opening in which the pharmaceutical agent, or a sterile air stream with the agent entrained therein, is flowed through and discharged from the wand.

A fiber optic core 192 depicted partially in phantom, extends longitudinally through the wand body 172. Core 192 has a distal end face 194 that is located in an opening in body undercut surface 186. Core face 194 is typically parallel to, if not also flush with surface 186.

Wand 170 it can be positioned over a surgical site in a manner similar to that in which wand 132 is positioned. The sterile air discharged from channel 178 forms a laminar barrier-defining air flow over the site. A pharmaceutical agent is discharged directly into the site through channel 178 and opening 190. Light is emitted over the site from the distal end face 194 of core 192.

It should be appreciated that the foregoing is directed to specific versions of the system of this invention. Other versions of the invention may have features different from what has been described.

Thus, it should be understood that other versions of this invention may include tools different from the described retractor and suction wand. Some tools into which fluid stream conduits and/or energy emitters of this invention can be integrated into include: forceps; clamps; debriders; irrigators; universal handpieces (drills); scalpels; tweezers; broaches; reamers; implant trials; and speculums. Similarly, the retractors and suction wands of this invention may have designs different from what has been shown. Also, not all versions of the invention are required to be of the above described designs.

Devices other than inductors may be employed to blend material into the sterile gas stream that is used to form the primary component, by volume, of the fluid stream discharged from the system. Similarly other gases than oxygen and CO₂ may be added to the stream. Such gases include argon and nitrogen. In some embodiments of the invention, it may be desirable to form the gas stream out of blend of gases that are lighter than air. The plume of gas created by the discharge of this fluid stream traps viral and bacterial sized contaminates and carries them away from the surgical site. Such a gas blend can for example, include helium as a component.

Alternative means of providing light to activate photosensitive agents may also be mounted in the tools of this invention. Thus, some tools of this invention may be provided with one or more LEDs. These LEDs emit light at the wavelength appropriate to activate the photosensitive agent discharge by the tool. In these constructions of the invention, a power supply located away from the surgical site supplies the energization signal needed to actuate the LED(s). Alternatively, batteries may be mounted to the tool. The batteries provide the charge needed to energize the light emitting devices integral with the tool. An advantage of this construction of the invention is that it eliminates the need to have a power supply cable that extends from the tool.

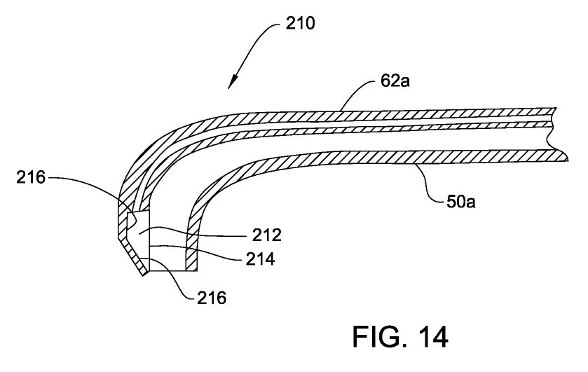

Furthermore, some tools of this invention may be constructed so that the light emitting device is arranged to emit light directly into the conduit integral with the tool through which the photodynamic agent is flowed prior to discharge from the tool. FIG. 14 illustrates the distal end of an alternative retractor 210 of this invention. Generally, retractor 210 has the same overall shape and proximal end features of retractor 22. The distal end retractor 210 is shaped so that internal to rib 62a there is lens 212. More particularly, lens 212 is fitted in a void space (not identified, at the distal end of the rib. This void space is contiguous with the elongated hollow conduit defined by the retractor main body 50a. Lens 212 has a face 214 that directed towards and forms part of the elongated hollow conduit. Retractor 210 is further formed so that the distal outer surfaces of lens 212 are coated with a reflective material, represented by lines 216.

Consequently, when retractor 210 of this version of the invention is employed, the light supplied to lens 212 reflects off the outer wall coatings. The light is then emitted outwardly through face 214 into the hollow conduit through which the fluid stream, including the photodynamic agent, is flowing. Thus, in this version of the invention, the photodynamic agent is activated before the agent is discharged from the tool 210. Activation of the photodynamic agent can be enhanced by adding oxygen to the gas stream flowed through and discharged from the tool.

In one alternative embodiment of the above-described version of the tool, the tool component that defines the conduit is a tube formed from an optically transmissive material. The outer surface of this tube is coated with a reflective material. Thus, when this tool is employed light is emitted throughout a substantial portion of the length of the conduit through which the photodynamic agent flows prior to discharge.

Other embodiments of this version of the invention may have different components that emit light to the fluid stream that flows through the tool conduit. Thus, in one embodiment of the invention, the light emitting component may be a ring formed of transparent material. The center of the ring defines part of the conduit through which the fluid stream flows prior to discharge. The outer surface of the ring is covered with reflective material so as to direct the light entering the ring towards the center, the conduit.

Another embodiment of the above-described version of the invention is constructed so that a surface of the conduit through which the sterile gas is flowed is coated with the photodynamic agent. This agent may be in a solid, gel or liquid state. Opposite this surface, the portion of the tool that defines the conduit is a light emitting component. This component may be a lens that emits light transmitted from another source or a light emitting device. When this embodiment of the invention is employed, the fluid stream that is discharged through the conduit includes oxygen. The light strikes the photodynamic agent so as to cause it to react with the oxygen. The light and oxygen activated photodynamic agent is then discharged as part of the fluid stream so that it can have desired therapeutic effect on the adjacent tissue.

Similarly, there is no requirement that all versions of the invention have each of the above-described features. Thus, there is no requirement that the inductors for mixing water or pharmaceutical agents be provided in all versions of the system. Likewise, there may be versions of the invention in which there is no need to provide the tools with a system for emitting light. Other components can be added to the system. For example, an oxygen concentrator can be provided so that the fluid stream discharged over the surgical site is oxygen rich. Also, a heater may be in line with the gas supply line 38. This heater warms the fluid stream prior to its discharge. The thermal energy given off by the fluid stream on discharge would thus counterbalance the loss of heat from the tissue due to the fact that the operating room tends to be a relatively cold environment.

Likewise, there is no requirement that in all versions of this invention, the energy-activated agent discharged from the tool be one that is activated in response to absorbing photonic (light) energy. In some versions of the invention, the agent may be one that is activated by other forms of energy. Thus, the agent may be contained microcapsules. The capsules are opened by the application of sonic energy. In these versions of the invention, the energy emitter attached to the tool is a transducer that emits sonic energy at a frequency that vibrates the microcapsules open.

It should be understood that devices other than the disclosed footswitch assembly can be used to regulate the various sub-assemblies of the system of this invention.

Also, the different components of this system can be recombined. For example, a lens and a proximally extending fiber optic cable can be attached to nozzle 124. This sub assembly is used to provide light for actuating any photosensitive pharmacological agent that is discharged with the gas forming the sterile barrier.

Therefore, it is an object of the appended claims to cover all such variations and modifications that come within the true spirit and scope of this invention.

What is claimed is:

1. A system for supplying and activating a therapeutic agent at a surgical site, said system including:
a source of therapeutic agent, the therapeutic agent being activated in response to the application of energy to the therapeutic agent;
a surgical tool for application to or adjacent a surgical site, said tool capable of performing a task other than the delivery or activation of the therapeutic agent, said tool being connecting to said therapeutic agent source and having a housing with : an internal bore through which the therapeutic agent is flowed; and a discharge port through which the therapeutic agent is discharged towards or within a surgical site; and
an energy emitter attached to said tool housing that emits energy capable of activating the therapeutic agent, said energy emitter positioned to direct energy towards the flow of the therapeutic agent.

2. The system of claim 1, wherein said energy emitter emits light energy.

3. The system of claim 1, wherein said energy emitter is attached to said tool housing to emit energy that is discharged from said tool housing.

4. The system of claim 1, wherein said energy emitter is attached to said tool housing to emit energy towards a location to which said surgical tool is applied.

5. The system of claim 1, wherein said one from the group consisting of: retractors; suction wands; forceps; clamps; debriders; irrigators; universal handpieces; scalpels; tweezers; broaches; reamers; femoral canal brush; implant trials; speculums; laser tools for tissue removal; tissue coagulation tools; RF tissue ablation tools; RF tissue cutting tools; and tools that emit ultrasonic energy for therapeutic purposes.

6. A surgical retractor, said retractor having:
a body including a foot shaped to hold tissue, said body being formed with: a fitting for receiving a fluid; a channel that extends from said fitting towards the body foot; and a discharge port in or adjacent the foot through which fluid is discharged from the channel; and
an energy emitter mounted to said body for emitting energy in the direction in which fluid is discharged from the foot.

7. The surgical retractor of claim 6, wherein said energy emitter emits photonic energy.
